(19) United States
(12) Patent Application Publication
(10) Pub. No.: US 2024/0115216 A1
(43) Pub. Date: Apr. 11, 2024

(54) POSITIONAL NON-CONTACT BABY MONITORING

(71) Applicant: Covidien LP, Mansfield, MA (US)

(72) Inventors: Paul S. ADDISON, Edinburgh (GB); Dean MONTGOMERY, Edinburgh (GB); Philip C. SMIT, Hamilton (GB); Rakesh K. SETHI, Vancouver (CA)

(21) Appl. No.: 18/455,528

(22) Filed: Aug. 24, 2023

Related U.S. Application Data
(60) Provisional application No. 63/379,101, filed on Oct. 11, 2022.

(51) Int. Cl. A61B 5/00; A61B 5/11; A61B 5/1171; G08B 3/10; G08B 5/22; G08B 21/02; G08B 21/04

(52) U.S. Cl. CPC A61B 5/746; A61B 5/0077; A61B 5/1113; A61B 5/1176; A61B 5/7405; A61B 5/742; G08B 3/10; G08B 5/22; G08B 21/0211; G08B 21/0415; A61B 2503/04

(57) ABSTRACT

Use of non-contact monitoring systems to monitor a subject (e.g., baby, infant, child) in a sleeping or resting environment. The systems identify the position of the subject and alert a caregiver if the subject is in a potentially hazardous position, such as lying prone (on their front) or if the subject has remained in the same position for a period of time longer than a threshold time. The non-contact monitoring systems can be configured to detect a physical attribute (e.g., respiration rate, tidal volume, pulse, etc.).

### CROSS-REFERENCE TO RELATED APPLICATIONS

The present application claims benefit of priority to U.S. Provisional Patent Application No. 63/379,101, entitled “POSITIONAL NON-CONTACT BABY MONITORING” and filed on Oct. 11, 2022, which is specifically incorporated by reference herein for all that it discloses or teaches.

### BACKGROUND

Baby monitors are well known. Such monitors typically have either or both video cameras and audio microphones that are placed in close proximity to the child which transmit a signal to a remote monitor (video and/or audio speaker) to provide the caregiver with a visual and/or audio signal. From the signal, the caregiver can determine if the child is uncomfortable or in distress. Unfortunately, the caregiver is advised of discomfort or distress by the audio emanating from the child. Typical monitors fail to provide any indication, other than a video image, regarding the position of the child.

It would be beneficial for a caregiver to know the position of the child, e.g., if the child is in a prone position (lying on the front face down), which is a contributing factor to Sudden Infant Death Syndrome (SIDS), without having to look at an image. Accordingly, there is a need for a better child monitoring system.

### SUMMARY

The present disclosure is directed to using non-contact monitoring systems to monitor the position of a subject (e.g., baby, infant, child) in a sleeping or resting environment. The systems utilize various measurement techniques to identify positions of the subject that might be potential hazards to the subject and alert a caregiver.

The non-contact monitoring systems incorporate the detection of the position or posture of a subject, such as an infant child. Should the subject be positioned in or move to a non-desirable or dangerous position, then an alert or alarm may be sounded. The systems may also indicate when a subject has lain too long in a certain position, e.g., whereby the development of bones and or other physiology may be impeded or has been placed in a dangerous position.

One particular embodiment described herein is a method of monitoring a subject. The method includes detecting a position of the subject with a non-contact monitoring system, determining, with the non-contact monitoring system, if the subject is in a prone position, and upon determining the subject is in a prone position, the non-contact monitoring system initiating an alarm.

Another particular embodiment described herein is another method of monitoring a subject with a non-contact monitoring system. The method includes detecting a subject in a region of interest (ROI) and determining a position of the subject, upon determining the position of the subject is a prone position, initiating a first alarm, upon determining the position of the subject is a supine position, monitoring the subject for movement for a time duration, and upon monitoring no movement within the time duration, initiating a second alarm.

Other embodiments are also described and recited herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

These and other aspects of the technology described herein will be apparent after consideration of the Detailed Description and Drawing herein. It is to be understood, however, that the scope of the claimed subject matter shall be determined by the claims as issued and not by whether given subject matter addresses any or all issues noted in the Background or includes any features or aspects recited in the Summary.

### BRIEF DESCRIPTION OF THE DRAWING

FIG. 1A is a simulated image of a monitored subject in an acceptable situation; FIG. 1B is a simulated image of the monitored subject in a position that is a potential hazard, the image indicating an alert; and FIG. 1C is a simulated image of the monitored subject showing another position that is a potential hazard to the subject, the image indicating an alert.

FIG. 2 is a schematic diagram of an example non-contact monitoring system.

FIG. 3 is a schematic diagram of another example non-contact monitoring system.

FIG. 4 is a block diagram of a non-contact monitoring system including a computing device, a server, and an image capture device according to various embodiments described herein.

FIG. 5 is a step-wise method for monitoring a subject.

FIG. 6 is another step-wise method for monitoring a subject.

### DETAILED DESCRIPTION

As described above, the present disclosure is directed to monitoring a subject (e.g., baby, infant, toddler) while resting or sleeping. Using the non-contact monitoring systems described herein mitigates the risk factors associated with position of the subject, such as for Sudden Infant Death Syndrome (SIDS) due to being in a prone position (lying on the front face down) and for flattening of the skull (plagiocephaly) due to the subject remaining in one position for extended periods of time. To prevent SIDS, parents are advised to keep infants on their back, however, this may cause flattening of the skull, either on the back of the skull or on the side of the skull, if the baby has a prevalence for turning its head in a certain direction. The systems can be used in a residential setting or in a medical or commercial setting, such as a hospital or other care facility.

The non-contact monitoring systems use a video signal of the subject, identifying physiologically relevant areas within the video image (such as the subject's head, face, neck, arms, legs, or torso) to determine the position of the subject. Using the video image, the systems extract a distance or depth signal from the relevant area, correlate the depth signals to the presence and position of the subject based on topographical mapping, optionally correlate any change in depth signals over time to movement of the subject, and use the position and movement to determine a potential threat to the subject. If a potential threat is detected, the systems issue an alert. Optionally, the physiological attributes of the subject may additionally be monitored by the systems, such as respiration (e.g., respiration rate, respiration/tidal volume), temperature, pulse, etc.

With the non-contact monitoring systems, signals representative of the topography and movement of the subject are detected by a camera or camera system that views but does not contact the subject. The camera or camera system may utilize any or all of depth signals, color signals (e.g., RGB signals), and IR signals. Facial recognition software may be used to confirm facial features. With appropriate selection and filtering of the signals detected by the camera, the physiologic contribution by each of the detected signals can be isolated and measured.

Remote sensing of a subject with video-based monitoring systems, in general, often presents several challenges. One challenge is ambient light. In this context, “ambient light” means surrounding light not emitted by components of the camera or the monitoring system. In some embodiments of the non-contact monitoring system, the desired physiologic signal is generated or carried by a light source. Thus, because of this, the ambient light cannot be entirely filtered, removed, or avoided as noise. Changes in lighting within the room, including overhead lighting, sunlight, television screens, nightlights, variations in reflected light, and passing shadows from moving objects all contribute to the light signal that reaches the camera. Even subtle motions outside the field of view of the camera can reflect light onto the subject being monitored.

The present disclosure describes methods of non-contact monitoring of a subject to determine the position of the subject and alert of potential hazards due to the subject's position. The methods are particularly useful for alerting caregivers (e.g., parents) of a subject's (e.g., child's, e.g., infant's) position in bed (e.g., lying prone, which increases the probability of re-breathing exhaled breather leading to carbon dioxide buildup and low oxygen levels, of upper airway obstruction, and of interfering with body heat dissipation leading to overheating, all which increase the likelihood of SIDS) and duration of a subject's time duration in a particular position. The methods may also monitor physiological conditions of the subject and alert of potential hazards.

The non-contact monitoring systems used for the non-contact monitoring of the subject are developed to identify features of the subject to determine the physical position of the subject and to determine and monitor the time duration the subject is in one position. Upon determining a potential threat, the systems provide an alert to the caregiver. The alerts can be adjusted or modified for the particular subject (e.g., by the caregiver, pursuant to advice from a physician).

The non-contact systems receive a video signal from the subject and the environment and from that extract a distance or depth signal from the relevant area to provide a topographical map from the depth signal; the systems may also determine any movement or motion from the depth signal. The systems can also receive a second signal, a light intensity signal reflected from the subject and environment, and from the reflected light intensity signal calculate a depth or distance and also a movement or motion. In some embodiments, the light intensity signal is a reflection of a pattern or feature (e.g., using visible color or infrared) projected onto the subject, such as by a projector.

The depth sensing feature of the system provides a measurement of the distance or depth between the detection system and the subject. One or two video cameras may be used to determine the depth, and change in depth, from the system to the subject. When two cameras, set at a fixed distance apart, are used, they offer stereo vision due to the slightly different perspectives of the scene from which distance information is extracted. When distinct features are present in the scene, the stereo image algorithm can find the locations of the same features in the two image streams. However, if an object is featureless (e.g., a smooth surface with a monochromatic color), then the depth camera system may have difficulty resolving the perspective differences. By including an image projector to project features (e.g., in the form of dots, pixels, etc., visual or IR) onto the scene, this projected feature can be monitored over time to produce an estimate of location and any change in location of an object.

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

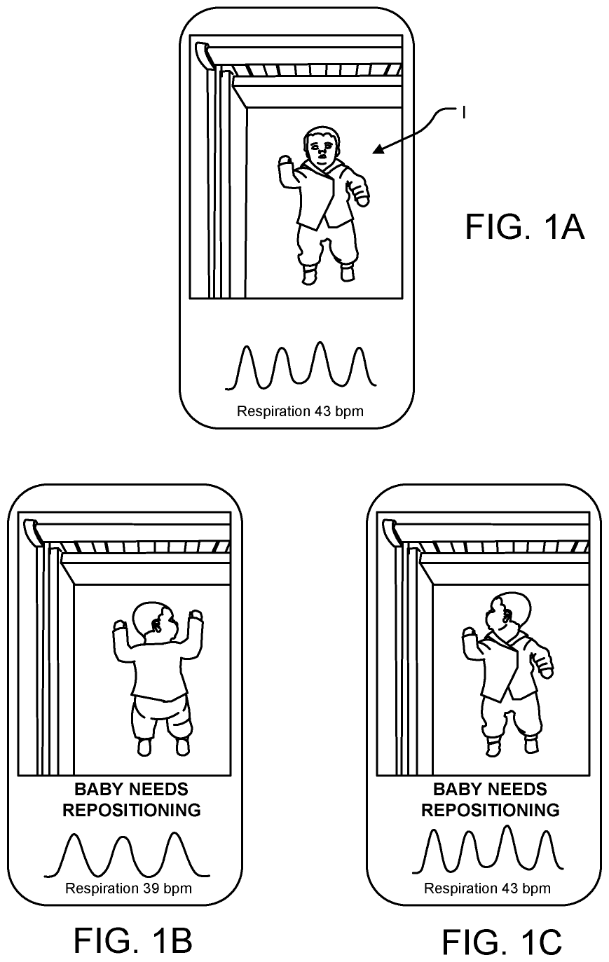

FIGS. 1A, 1B and 1C show example simulated images from non-contact monitoring systems of this disclosure, the images illustrating various situations detectable by the systems. The images are simulated images on a device such as a cell phone or tablet showing a subject I (particularly in these images, an infant) in a crib.

In FIG. 1A, the infant is seen centered in the crib lying supine (on its back). In this embodiment of the visual provided by the system, the subject's respiration rate is also provided in the image, both in graphical form and numerical. Not shown, but the respiration may be indicated by a color superimposed on the torso,

In FIG. 1B, the infant is seen in the crib lying in a prone position (on its front). The system has been programmed to recognize that at least the subject's head and/or face are no longer identifiable or that the face profile is identified, e.g., by using the color or IR image or through the topography determined using the depth camera, and/or using facial recognition software, e.g., artificial intelligence. A warning is provided in the image by the system alerting the caregiver that the subject should be repositioned. The alert may be, e.g., flashing, red or orange in color, or have any other features to draw the caretaker's attention to it. Additionally, an audible warning (alert) may be issued by the system. The subject's respiration rate is also provided in the image; with the subject lying in a prone position, the respiration rate has decreased related to FIG. 1A and the supine position. In some embodiments, the system may issue an alert based on the respiration rate either exceeding a threshold or falling below a threshold, regardless of the subject's position.

In FIG. 1C, the infant is seen in the crib lying in a supine position, with the head turned to the right. The system has been programmed to recognize that the subject's head is turned, due to, e.g., the ear being identified or the face profile identified, e.g., by using the color or IR image, through the topography determined using the depth camera, and/or using facial recognition software, e.g., artificial intelligence. The system includes a timer to track the time duration that the subject remains in this position, e.g., the time duration without movement. If the subject has a prevalence for turning its head in a certain direction for an extended period of time, it may cause flattening of that side of the skull. Thus, a warning is provided in the image by the system alerting the caregiver that the subject should be repositioned if the duration in the particular position exceeds a predetermined threshold. The alert may be, e.g., flashing, red or orange in color, or have any other features to draw the caretaker's attention to it. The warning period, in some embodiments, may be adjustable by the caretaker, pursuant to a physician's advice. Additionally, an audible warning (alert) may be issued by the system. The subject's respiration rate is also provided in the image.

The system may warn if the subject is in other positions for a period of time longer than the predetermined threshold. For example, as an infant ages, side sleep may be acceptable, however, if too long in the same position, a warning may be issued.

In both situations, FIG. 1B and FIG. 1C, the system issues an alarm or alert to the caregiver to reposition the subject. In some embodiments, e.g., as the subject ages, the time period threshold for issuing an alert may change; the change may be programmed into the system or may be manually entered by a caregiver e.g., pursuant to advice from a physician. For example, as an infant ages, the acceptable time duration spent with the head turned one direction can increase. Additionally, as the subject ages, e.g., is a toddler, a period of time in the prone position may be acceptable, pursuant to advice from a physician. The system may merely monitor for time duration exceeding a threshold in a position without movement, regardless of if in the prone or supine position.

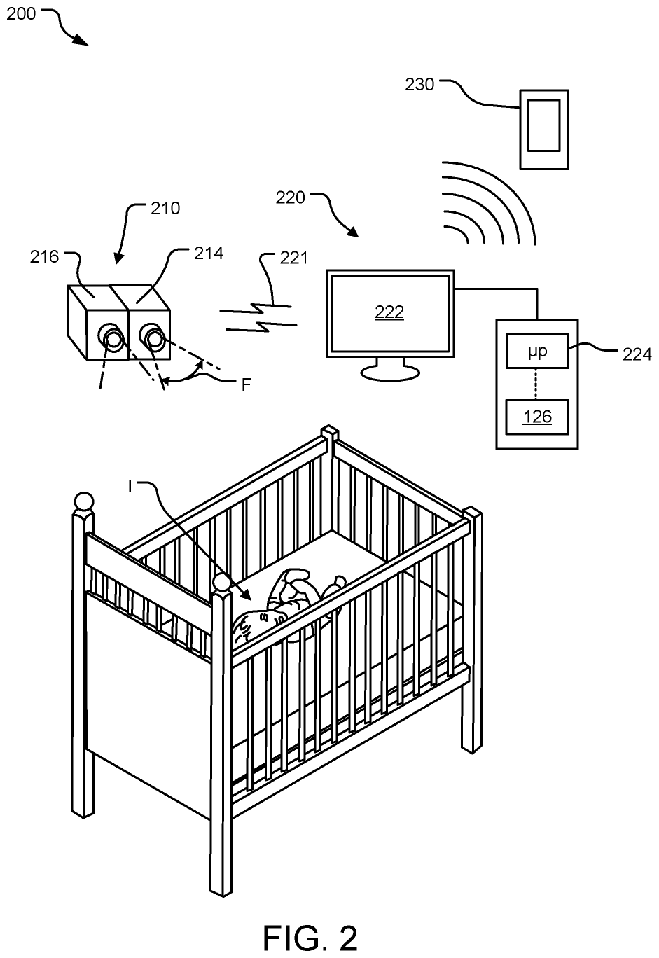

FIG. 2 shows a non-contact subject monitoring system 200 and a subject I, in this particular example an infant in a crib. It is noted that the systems and methods described herein are not limited to a crib, but may be used with a bassinette, an isolette, or any other place where the subject may be left alone, in a residential or commercial setting. The system 200 includes a non-contact detector system 210 placed remote from the subject I. In this embodiment, the detector system 210 includes a camera system 214, particularly, a camera that includes an infrared (IR) detection feature. The camera 214 may be a depth sensing camera, such as a Kinect camera from Microsoft Corp. (Redmond, Washington) or a RealSense™ D415, D435 or D455 camera from Intel Corp. (Santa Clara, California). The camera system 214 is remote from the subject I, in that it is spaced apart from and does not physically contact the subject I. The camera system 214 may be positioned in close proximity to or on the crib. The camera system 214 includes a detector exposed to a field of view F that encompasses at least the head/face portion of the subject I. Because infants and toddlers are notorious for moving, the field of view F generally encompasses the entire sleeping (e.g., crib) area.

The camera system 214 includes a depth sensing camera that can detect a distance between the camera system 214 and objects in its field of view F. Such information can be used, as disclosed herein, to determine that a subject is within the field of view of the camera system 214 and determine a region of interest (ROI) to monitor on the subject. Once an ROI is identified, that ROI can be monitored over time and the depth data can be used to determine the position of the subject I and a change in depth of points can represent movements of the subject I. The field of view F is selected to be at least the head/neck and optionally upper torso of the subject. However, as it is common for young children and infants to move within the confines of their crib, bed or other sleeping area, the entire area potentially occupied by the subject I (e.g., the crib) may be the field of view F. The ROI may be the entire field of view F or may be less than the entire field of view F.

The camera system 214 may operate at a set frame rate, which is the number of image frames taken per second (or other time period). Example frame rates include 20, 30, 40, 50, or 60 frames per second, greater than 60 frames per second, even less than 20 frames per second, and any values between those. Frame rates of 20-30 frames per second produce useful signals, though frame rates above 100 or 120 frames per second are helpful in avoiding aliasing with light flicker (for artificial lights having frequencies around 50 or 60 Hz).

The distance from the ROI on the subject I to the camera system 214 is measured by the system 200. Generally, the camera system 214 detects a distance between the camera system 214 and the surface within the ROI; the change in depth or distance of the ROI can represent movements of the subject I.

In some embodiments, the system 200 determines a skeleton outline of the subject I to identify a point or points from which to extrapolate the ROI. For example, a skeleton may be used to find a center point of a chest, shoulder points, waist points, hands, head, and/or any other points on a body. These points can be used to determine the ROI. In other embodiments, instead of using a skeleton, other points are used to establish an ROI. For example, a face may be recognized, and a torso and waist area inferred in proportion and spatial relation to the face.

In another example, the subject I may wear a specially configured piece of clothing that identifies points on the body such as the torso or the arms. The system 200 may identify those points by identifying the indicating feature of the clothing. Such identifying features could be a visually encoded message (e.g., bar code, QR code, etc.), or a brightly colored shape that contrasts with the rest of the subject's clothing, etc. In some embodiments, a piece of clothing worn by the subject may have a grid or other identifiable pattern on it to aid in recognition of the subject and/or their movement. In some embodiments, the identifying feature may be stuck on the clothing using a fastening mechanism such as adhesive, a pin, etc., or stuck directly on the subject's skin, such as by adhesive. For example, a small sticker or other indicator may be placed on a subject's hands that can be easily identified from an image captured by a camera.

In some embodiments, the system 200 may receive a user input to identify a starting point for defining an ROI. For example, an image may be reproduced on an interface, allowing a user of the interface to select a point on the subject from which the ROI can be determined (such as a point on the head). Other methods for identifying a subject, points on the subject, and defining an ROI may also be used.

However, if the ROI is essentially featureless (e.g., a smooth surface with a monochromatic color, such as a blanket or sheet covering the subject I), then the camera system 214 may have difficulty resolving the perspective differences. To address this, the system 200 can include a projector 216 to project individual features (e.g., dots, crosses or Xs, lines, individual pixels, etc.) onto objects in the ROI; the features may be visible light, UV light, infrared (IR) light, etc. The projector may be part of the detector system 210 or the overall system 200.

The projector 216 generates a sequence of features over time on the ROI from which is monitored and measured the reflected light intensity. A measure of the amount, color, or brightness of light within all or a portion of the reflected feature over time is referred to as a light intensity signal. The camera system 214 detects the features from which this light intensity signal is determined. In an embodiment, each visible image projected by the projector 216 includes a two-dimensional array or grid of pixels, and each pixel may include three color components—for example, red, green, and blue (RGB). A measure of one or more color components of one or more pixels over time is referred to as a “pixel signal,” which is a type of light intensity signal. In another embodiment, when the projector 216 projects an IR feature, which is not visible to a human eye, the camera system 214 includes an infrared (IR) sensing feature. In another embodiment, the projector 216 projects a UV feature. In yet other embodiments, other modalities including millimeter-wave, hyper-spectral, etc., may be used.

The projector 216 may alternately or additionally project a featureless intensity pattern (e.g., a homogeneous, a gradient or any other pattern that does not necessarily have distinct features, or a pattern of random intensities). In some embodiments, the projector 216, or more than one projector, can project a combination of a feature-rich pattern and featureless patterns on to the ROI.

The light intensity of the image reflected by the subject surface is detected by the detector system 210.

The measurements (e.g., depth signal, RGB reflection, light intensity) are sent to a computing device 220 through a wired or wireless connection 221. The computing device 220 includes a display 222, a processor 224, and hardware memory 226 for storing software and computer instructions. Sequential image frames of the subject I are recorded by the video camera system 214 and sent to the computing device 220 for analysis by the processor 224. The display 222 may be remote from the computing device 220, such as a video screen positioned separately from the processor and memory. Other embodiments of the computing device 220 may have different, fewer, or additional components than shown in FIG. 2. In some embodiments, the computing device may be a server. In other embodiments, the computing device of FIG. 2 may be connected to a server. The captured images (e.g., still images or video) can be processed or analyzed at the computing device and/or at the server to create a topographical map or image to identify the subject I and any other objects with the ROI.

In some embodiments, the computing device 220 is operably connected (e.g., wirelessly, via WiFi connectivity, cellular signal, Bluetooth™ connectivity, etc.) to a remote device 230 such as a smart phone, tablet, or merely a screen. The remote device 230 can be remote from the computing device 220 and the subject I, for example, in an adjacent or nearby room. The computing device 220 may send a video feed to the remote device 230, showing e.g., the subject I and/or the field of view F. The image may be, e.g., a depth data feed, an RGB feed or a dark IR feed. The image may include a thermal feed with false coloring according to temperature and/or a combination of these imaging modalities. Additionally or alternately, the computing device 220 may send instructions to the remote device 230 to trigger an alarm, such as when the system 200 detects that a subject is in a problematic position, in a position for too long of a time duration, or if a physiological attribute is above or below a threshold.

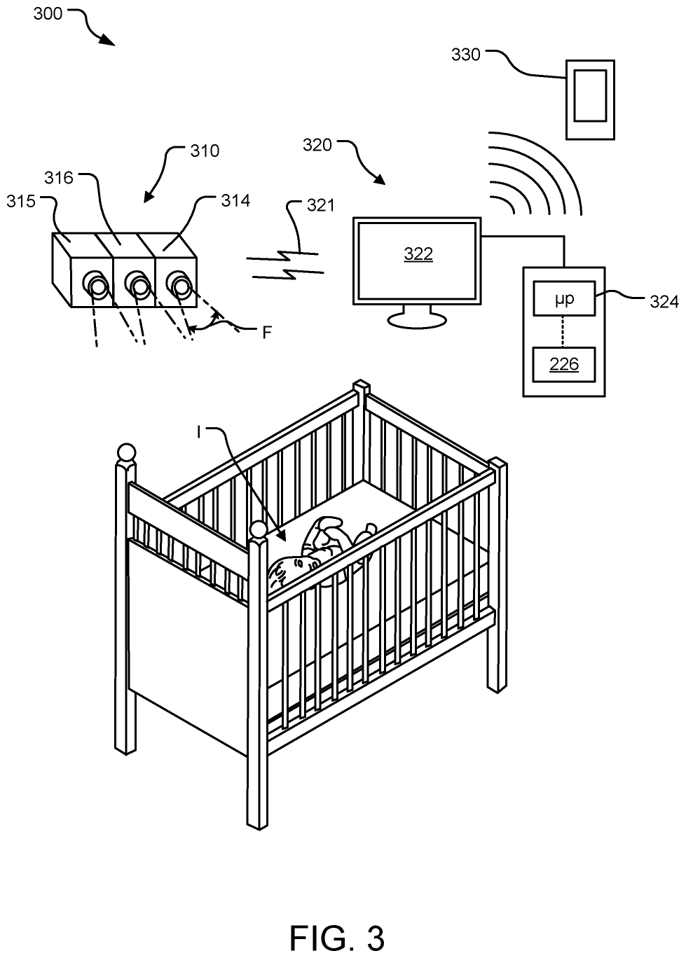

FIG. 3 shows another non-contact subject monitoring system 300 and a subject I, in this example, an infant in a crib. The system 300 includes a non-contact detector 310 placed remote from the subject I. In this embodiment, the detector 310 includes a first camera 314 and a second camera 315, at least one of which includes an infrared (IR) camera feature. The cameras 314, 315 are positioned so that their ROIs at least intersect, in some embodiments, completely overlap. The detector 310 also includes an IR projector 316, which projects individual features (e.g., dots, crosses or Xs, lines, or a featureless pattern, or a combination thereof etc.) onto the subject I in the ROI. The projector 316 can be separate from the detector 310 or integral with the detector 310, as shown in FIG. 3. In some embodiments, more than one projector 316 can be used. The projector 316 can be switched on continuously or, for multiple projectors 316, can be temporally multiplexed (e.g., so that they are not switched on at the same moment, or, there may be some overlap between when the projectors 316 are switched on and off). Both cameras 314, 315 are aimed to have features projected by the projector 316 to be in their ROI. The cameras 314, 315 and projector 316 are remote from the subject I, in that they are spaced apart from and do not contact the subject I. In this implementation, the projector 316 is physically positioned between the cameras 314, 315, whereas in other embodiments it may not be so.

The distance from the ROI to the cameras 314, 315 is measured by the system 300. Generally, the cameras 314, 315 detect a distance between the cameras 314, 315 and the projected features on a surface within the ROI. The light from the projector 316 hitting the surface is scattered/diffused in all directions; the diffusion pattern depends on the reflective and scattering properties of the surface. The cameras 314, 315 also detect the light intensity of the projected individual features in their ROIs. From the distance and the light intensity, the presence and position of the subject I is monitored, as well as any movement of the subject I.

The detected images, diffusion measurements and/or reflection pattern are sent to a computing device 320 through a wired or wireless connection 321. The computing device 320 includes a display 322, a processor 324, and hardware memory 326 for storing software and computer instructions. The display 322 may be remote from the computing device 320, such as a video screen positioned separately from the processor and memory. In other embodiments, the computing device of FIG. 3 may be connected to a server. The captured images (e.g., still images or video) can be processed or analyzed at the computing device and/or at the server to create a topographical map or image to identify the subject I and any other objects with the ROI.

In some embodiments, the computing device 320 is operably connected (e.g., wirelessly, via WiFi connectivity, cellular signal, Bluetooth™ connectivity, etc.) to a remote device 330 such as a smart phone, tablet, or merely a screen. The remote device 330 can be remote from the computing device 320 and the subject I, for example, in an adjacent or nearby room. The computing device 320 may send a video feed to the remote device 330, showing, e.g., the subject I and/or the field of view F. Additionally or alternately, the computing device 320 may send instructions to the remote device 230 to trigger an alarm, such as when the system 300 detects that the subject I is in a problematic position, in a position for too long of a time duration, or if a physiological attribute is above or below a threshold causing a potential hazard to the subject I.

The computing device 220, 320 determines, from the image of the ROI (formed from the, e.g., depth signal, RGB reflection, light intensity measurements), the position of the head and/or face of the subject. The computing device 220, 320 then determines, from the image, if the face or side of the head/face is identifiable, thus determining whether the subject's face is upright (indicating lying on one's back) or to the side (indicating either lying one one's back with the head turned or lying on one's front with the head turned). If the side of the face is identified, various details of the subject's body positioning are evaluated to determine if the subject is lying on the back with the head turned or lying on the front with the head turned. If it is identified that the subject is on their front (see, e.g., FIG. 1B), the computing device 220, 320 initiates an alarm on the remote device 230, 330. If is it identified that the subject is on their back, a timer is initiated to measure the time in that position. Upon reaching a predetermined threshold time at that position, the computing device 220, 320 initiates an alarm on the remote device 230, 330 (see, e.g., FIG. 1C). Additionally, the computing device 220, 230 may initiate an alarm if a monitored physiological attribute (e.g., respiration rate, tidal volume, etc.) is above or below a predetermined threshold.

The non-contact monitoring systems 200, 300 can be configured to detect a physical attribute (e.g., respiration rate, tidal volume, pulse, etc.) by monitoring the change in depth of a region of the ROI over time. For example, the system 200, 300 can detect the rise and fall of the subject's chest, due to the change in depth data, which can be correlated to respiration rate. Integrating the respiration rate can provide a tidal volume. Similarly, detecting the rise and fall of a region such as a vein in the subject's neck or temple can be correlated to pulse.

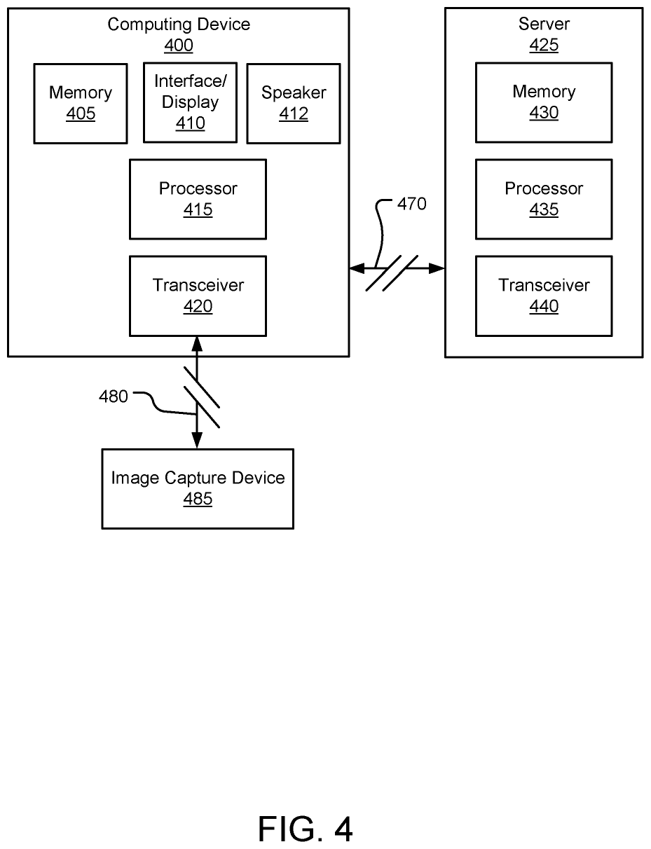

The computing device 220, 320 has an appropriate memory, processor, and software or other program to evaluate the ROI image, identify features, operate a timer, and determine if any of the monitored situations are potential hazards. FIG. 4 is a block diagram illustrating a system including a computing device 400, a server 425, and an image capture device 485 (e.g., a camera, e.g., the camera system 214 or cameras 314, 315). In various embodiments, fewer, additional and/or different components may be used in the system.

The computing device 400 includes a processor 415 that is coupled to a memory 405. The processor 415 can store and recall data and applications in the memory 405, including applications that process information and send commands/signals according to any of the methods disclosed herein. The processor 415 may also display objects, applications, data, etc. on an interface/display 410 and/or provide an audible alert via a speaker 412. The processor 415 may also or alternately receive inputs through the interface/display 410. The processor 415 is also coupled to a transceiver 420. With this configuration, the processor 415, and subsequently the computing device 400, can communicate with other devices, such as the server 425 through a connection 470 and the image capture device 485 through a connection 480. For example, the computing device 400 may send to the server 425 information determined about a subject from images captured by the image capture device 485, such as depth information of a subject or change in depth information over time.

The server 425 also includes a processor 435 that is coupled to a memory 430 and to a transceiver 440. The processor 435 can store and recall data and applications in the memory 430. With this configuration, the processor 435, and subsequently the server 425, can communicate with other devices, such as the computing device 400 through the connection 470.

The computing device 400 may be, e.g., the computing device 220 of FIG. 2 or the computing device 320 of FIG. 3. Accordingly, the computing device 400 may be located remotely from the image capture device 485, or it may be local and close to the image capture device 485 (e.g., in the same room). The processor 415 of the computing device 400 may perform any or all of the various steps disclosed herein. In other embodiments, the steps may be performed on a processor 435 of the server 425. In some embodiments, the various steps and methods disclosed herein may be performed by both of the processors 415 and 435. In some embodiments, certain steps may be performed by the processor 415 while others are performed by the processor 435. In some embodiments, information determined by the processor 415 may be sent to the server 425 for storage and/or further processing.

The devices shown in the illustrative embodiment may be utilized in various ways. For example, either or both of the connections 470, 480 may be varied. For example, either or both the connections 470, 480 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 470, 480 may be a dock where one device may plug into another device. As another example, one or both of the connections 470, 480 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 470, 480 may represent several different computing devices and network components that allow the various devices to communicate through the internet, either through a hard-wired or wireless connection. One or both of the connections 470, 480 may also be a combination of several modes of connection.

The configuration of the devices in FIG. 4 is merely one physical system on which the disclosed embodiments may be executed. Other configurations of the devices shown may exist to practice the disclosed embodiments. Further, configurations of additional or fewer devices than the ones shown in FIG. 4 may exist to practice the disclosed embodiments. Additionally, the devices shown in FIG. 4 may be combined to allow for fewer devices than shown or separated such that more than the three devices exist in a system. It will be appreciated that many various combinations of computing devices may execute the methods and systems disclosed herein. Examples of such computing devices may include other types of infrared cameras/detectors, night vision cameras/detectors, other types of cameras, radio frequency transmitters/receivers, smart phones, personal computers, servers, laptop computers, tablets, RFID enabled devices, or any combinations of such devices.

The non-contact monitoring systems and methods of this disclosure utilize depth (distance) information between the camera(s) and a subject to determine the position of the subject. The systems are programmed to recognize different positions and their possibility of posing a threat.

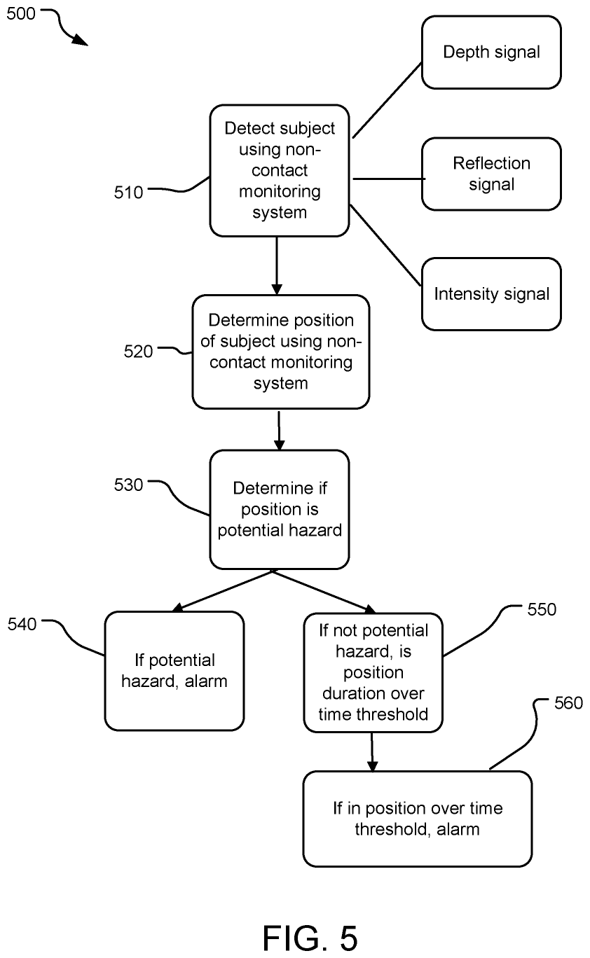

FIG. 5 provides a method 500 for monitoring a subject (e.g., infant, child) using a non-contact monitoring system as described herein. In a first step 510, the location of the subject is detected with the non-contact monitoring system utilizing depth information; the depth may be determined from depth measurements, from reflected signals, or from reflected intensity signals, for light, IR, RGB, etc. In a second step 520, the position of the subject is determined with the non-contact monitoring system utilizing the topography identified by the system, and in step 530, the non-contact monitoring system determines if the position is a potential hazard. Upon determining the subject is in a position that is a potential hazard to the subject, in step 540, the non-contact monitoring system initiates an alarm.

Subsequent or simultaneously, upon determining the subject is not in a position that is a potential hazard to the subject, the system monitors for movement in step 550 to determine if the subject is in the same position for a time duration over a threshold. If no movement is detected and the duration in the position is over the threshold time period, the non-contact monitoring system initiates an alarm in step 560.

In some embodiments, the system may monitor the subject only for a potentially hazardous position, no matter how long the subject is in an acceptable position, and in other embodiments the system may monitor only for movement within a predetermined time period, no matter what the subject's position.

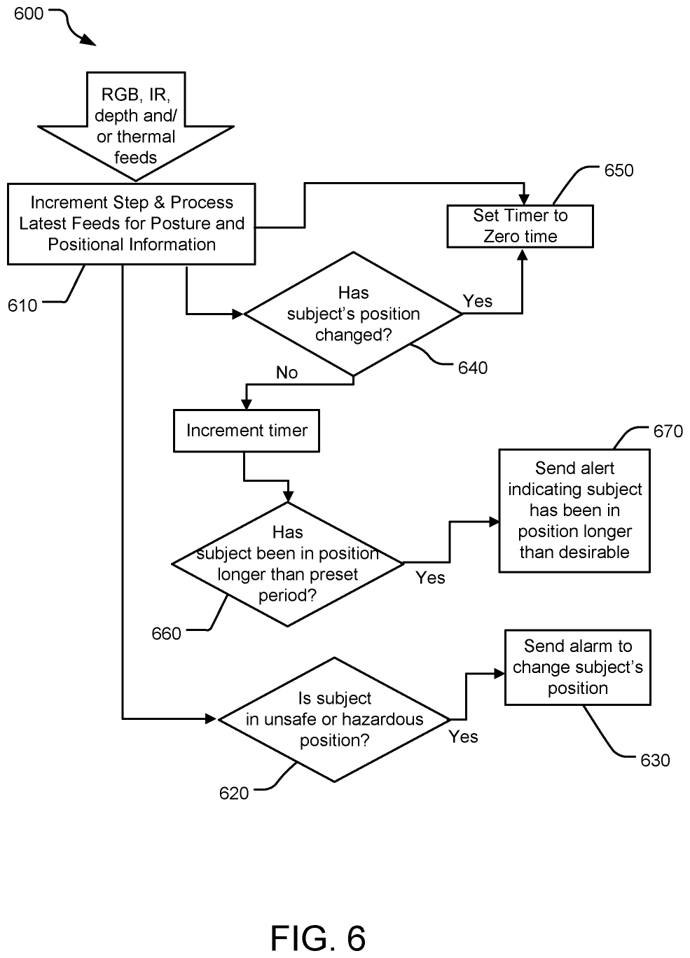

FIG. 6 provides a method 600 for monitoring a subject (e.g., baby) using a non-contact monitoring system as described herein. In a first step 610, the position of the subject is detected in incremental time periods (per a timer) with the non-contact monitoring system utilizing the RGB, IR, depth, and any other depth measurement feeds. In a step 620, the posture/position is analyzed to determine if the subject is in an unsafe position (e.g., on its front). If YES in step 620, an alarm is sent in step 630 to have the caretaker (e.g., parent) change the subject's position.

In a step 640 parallel to step 620, if the subject's position has changed in the time period, then in a subsequent step 650 the timer is reset and the position of the subject is again detected in step 610. If the subject's position has not changed in step 640, then the time period between the incremental time periods and a threshold period is compared. In step 660, it is determined if the subject has been in that position for longer than the threshold period. If YES in step 660, an alarm is sent in step 670 indicating the subject has been in the position longer than desired and to have the caretaker change the subject's position.

As indicated above, in addition to the methodology of this disclosure utilizing depth (distance) information between the camera(s) and the subject to determine presence and position of a subject, the method can also use reflected light intensity from projected light features and/or IR features (e.g., dots, grid, stripes, crosses, squares, etc., or a featureless pattern, or a combination thereof) in the scene to estimate the depth (distance). From the depth information, the position of the subject can be identified and the system can determine whether or not the position warrants an alarm to a caregiver.

The above specification and examples provide a complete description of the structure and use of exemplary embodiments of the invention. The above description provides specific embodiments. It is to be understood that other embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

1. A method of monitoring a subject, the method comprising:
detecting a position of the subject with a non-contact monitoring system;
determining, with the non-contact monitoring system, if the subject is in a prone position; and
upon determining the subject is in a prone position, initiating an alarm.

2. The method of claim 1, wherein detecting a position of the subject is via one or more of depth measurements, IR image reflection intensity, RGB reflection intensity, or facial recognition software.

3. The method of claim 1, further comprising:
upon determining the subject is not in a prone position, monitoring the subject for movement for a period of time.

4. The method of claim 3, upon determining no movement of the subject during the period of time, initiating an alarm.

5. The method of claim 1, wherein initiating an alarm comprises:
providing a visual alarm on a display.

6. The method of claim 5, wherein providing a visual alarm on a display comprises:
providing the visual alarm on a remote device.

7. The method of claim 1, wherein initiating an alarm comprises:
providing an audible alarm.

8. The method of claim 7, wherein providing an audible alarm comprises:
providing the audible alarm from a device remote from the subject.

9. A method of monitoring a subject with a non-contact monitoring system, the method comprising:
detecting a subject in a region of interest (ROI);
determining a position of the subject;
upon determining the position of the subject is a prone position, initiating a first alarm;
upon determining the position of the subject is a supine position, monitoring the subject for movement for a time duration; and
upon monitoring no movement within the time duration, initiating a second alarm.

10. The method of claim 9, wherein monitoring the subject for movement for a time duration comprises initiating a timer and resetting the timer upon monitoring movement.

11. The method of claim 10, wherein the time duration is based on age of the subject.

12. The method of claim 9, wherein initiating a first alarm and initiating a second alarm comprises:
initiating a visual alarm.

13. The method of claim 9, wherein initiating a first alarm and initiating a second alarm comprises:
initiating an audible alarm.

14. The method of claim 9, wherein initiating a first alarm and initiating a second alarm comprises:
initiating the first alarm and the second alarm on a device remote form the non-contact monitoring system.
